(19) United States
(12) Patent Application Publication
(10) Pub. No.: US 2019/0314287 A1
(43) Pub. Date: Oct. 17, 2019

(54) Method and System for Conditioning Lipid Compounds for Medicinal Foods and Medicinal Products

(71) Applicants: Belles Feuilles, Paris (FR); Cabinet du Docteur Bernard Kurdyk, Paris (FR)

(72) Inventors: Bernard Jacques Kurdyk, Paris (FR); Jean-Pierre Lablanchy, Paris (FR)

(21) Appl. No.: 16/473,175

(22) PCT Filed: Dec. 21, 2017
(86) PCT No.: PCT/EP2017/084259

§ 371 Date: Jun. 24, 2019

(30) Foreign Application Priority Data
Dec. 26, 2016 (FR) 1663398

(51) Int. Cl. A61K 9/48; A23P 10/30; A23L 33/105

(52) U.S. Cl. CPC A61K 9/4891; A23P 10/30; A23L 33/105; A61K 9/485; A61K 9/4875; A61K 9/4833

(57) ABSTRACT

A method for packaging a lipid compound for ingestion by a human or animal body, comprising the following steps:
filling (I) a first part of a prefabricated capsule, previously opened in two parts, with a predetermined volume of a lipid compound;
introducing (II) a food-grade lipophilic absorbent into the lipid compound (4) within this first part, so as to produce, after absorption of the lipid compound, a core in the form of a pasty mixture providing for a predetermined dissolution rate,
closing (III) the capsule by fitting the second part on the first part containing the core; and
coating the capsule thus closed with a polymer, so as to allow subsequent release of the lipid compound at a predetermined release site within said human or animal body.

### TECHNICAL FIELD

This invention concerns a method for packaging lipid compounds for functional foods or medicaments. It also covers a packaging system using this method.

Such a method is particularly aimed at packaging lipid complexes, particularly essential oils, in the agri-food and pharmaceutical fields.

### PRIOR ART

Solid lipophilic preparations can nowadays be manufactured by coating or encapsulation processes. The advantage is that these oily compounds can be transported and easily administered. This concerns the food industry (confectionery, flavor enhancers, etc.) but also the pharmaceutical industry.

Different technologies allow this coating of lipidic substances to be obtained, said technologies ensuring the production of final particles of very varied dimensions.

Thus, lipidic compounds can be, for example:
incorporated into microparticles with a liquid or solid core,
dispersed in droplets,
stabilized by crystallization, gelation, polymerization or precipitation.

The methods used include, for example, gelation, spray drying, prilling (micro-granulation), coating and emulsion techniques.

These techniques, well described and developed today, are dependent on the lipid compounds to be processed:
if concentrated oils are to be processed, their coating within a solid voluminous core ensures long-term preservation;
if low-concentrated oils are to be processed, an external, low-volume englobing means will ensure stability.

Thus, these technologies offer no solution for the incorporation of large volumes of high-concentration oils. The controlled release of these oil complexes, i.e. their release at a chosen place or time, is a major research and development issue in this field.

The intestinal absorption process, which remains variable and dependent on biological and/or galenic factors, will be recalled below. Biological factors include mechanical factors: gastric emptying rate and duration of intestinal transit, and chemical factors: composition and pH of digestive fluids.

The intestinal transit time varies according to the type of food, the type of subject and the conditions of intake. The kinetics of this transit can be estimated as follows:

| | | Intestine | | |
|---|---|---|---|---|
| Location | Stomach | Duodenum | Jejunum | Ileum |
| Time | Liquids and small | 5 to 15 | 120 to | 180 to |
| Minutes | quantities: 10 to 30 | | 210 | 360 |
| | Consistent food: 60 to 240 | | | |

The presence of food activates intestinal transit. A thick consistency slows down transit by increasing the residence time in the stomach. This underlines the importance of taking a glass of water with substances that are to be quickly absorbed.

In the chemical composition of digestive fluids, the most important factor is pH. This pH variation along the gastrointestinal tract is used to develop enteric forms.

| | | Intestine | | | |
|---|---|---|---|---|---|
| | | | Duodenum | | |
| | | Duodenum | Descending | | |
| Location | Stomach | Ampulla | part | Jejunum | Ileum |
| pH | 1.7 to 1.9 | 4 to 5 | 5 to 6 | 6 to 7 | 7 to 8 |

For oral pharmaceutical forms, bioavailability varies according to the chosen dosage form, and for the same form, it depends on the formulation and technology used.

Before being absorbed, the active ingredient must be solubilized in the digestive fluids. Before this dissolution step, the pharmaceutical form undergoes several modifications. The evolution is more or less complex depending on the form considered. For enteric coated tablets, release is delayed. The place of release is thus programmed, the release taking place only at the level of the intestine.

Currently, the coating processes for essential oils are carried out by creating a thick membrane, flexible or not, around the liquid core. The release of oils is done during ingestion when the patient crunches the capsule with the disadvantage of an often unpleasant taste in the mouth, or when the membrane is diluted in the stomach by gastric juices.

The purpose of this invention is to remedy this situation by proposing a method for packaging lipid components to create a container around a volume of essential oils, or a mixture or complex of essential oils. This container must be able to dissolve in the digestive tract to ensure the release of the oils in the desired area: stomach, anterior part of the digestive tract or colon.

The objective of this invention is also to make ingestion easy for patients in order to optimize dosage compliance and benefit from the therapeutic contributions.

### DESCRIPTION OF THE INVENTION

The above objective is achieved by a method for packaging a lipid compound intended to be ingested by a human or animal body, comprising the following steps:
filling a first part of a prefabricated capsule previously opened in two parts, with a predetermined volume of a lipid compound;
inserting a food grade lipophilic absorbent into the lipid compound within said first part, so as to provide, upon absorption of said lipid compound, a core in the form of a pasty mixture providing a predetermined dissolution rate,
closing said capsule by placing the second part on the first part containing said core; and
coating said capsule thus closed with a polymer, so that the lipid compound can be released at a predetermined release site within the human or animal body.

The coating step can advantageously include one or more polymer applications on the capsule the number of these applications determining the thickness of the coating layer and thus the capsule's resistance to acid attacks.

In a first version of the capsule prepared by the packaging method according to the invention, the coating polymer has a dissolution pH of approximately 6, so that the capsule is intended to release the lipid compound substantially in the duodenum.

In a second version of the capsule prepared by the packaging method according to the invention, the coating polymer has a pH substantially greater than or equal to 7, so that the capsule is intended to release the lipid compound in the remote regions of the digestive tract.

In a first particular embodiment of the packaging method according to the invention, the lipophilic absorbent comprises edible clay, so that the mixture thus obtained is thick and provides a very slow release of the lipid compound.

In a second particular embodiment of the packaging method according to the invention, the lipophilic absorbent comprises tapioca, so that the mixture thus obtained is flexible and provides for a rapid release of the lipid compound.

The lipid compound may include a pure essential oil or a mixture of essential oils.

This new method for packaging lipid complexes, especially essential oils, allows:
therapeutic compliance of patients to be improved;
the place of release of essential oils within the intestinal tract to be controlled;
a lipid compound to be released at the desired time by dissolving its coating in a predetermined place and/or time.

The effects obtained with the packaging method according to the invention lie in particular in the quality of the delivery of oils in the chosen part of the digestive tract and in the ease of ingestion for the patient, which optimizes treatment compliance.

The specificity of the invention also lies in the selective choice of the lipophilic saturation agent and of the peripheral coating for a perfectly controlled speed and hence release site of the oils.

The packaging method according to the invention thus meets the requirements for the release of active ingredients (in the form of essential oil) at the stomach and digestive tract level.

It allows an impervious and durable coating to be created, taking into account pharmaceutical and religious requirements, to ensure a unique manufacturing process, independently from the composition of the core to be coated, and to provide industrial feasibility.

In this context, the invention is particularly aimed at facilitating compliance with medical prescriptions by producing capsules that are easy to handle and to absorb, and are stable over time. The components for the production of the capsules are chosen in order to comply with religious requirements and concerns.

According to yet another aspect of the invention, a system is proposed for packaging a lipid compound intended to be ingested by a human or animal body, implementing the method according to the invention, comprising:
means for filling a first part of a prefabricated and enteric capsule previously opened in two parts, with a predetermined volume of a lipid compound;
means for inserting a food-grade lipophilic absorbent into the lipid compound within said first part, so as to provide, upon absorption of said lipid compound, a core in the form of a pasty mixture providing a predetermined dissolution rate,
means for closing said capsule by placing the second part on the first part containing said core; and
means for coating said capsule thus closed with a polymer provided for releasing the lipid compound in a predetermined release location.

The coating means can be advantageously arranged to produce one or more coating layers of selected polymers, the number of these processings determining the thickness of the coating layer and thus the capsule's resistance to acid attacks.

The method and system of packaging lipid compounds according to the invention can be applied to the production of capsules proposed as functional foods or as medicaments based on essential oils. This invention has a series of applications in the medical field where the benefit from the therapeutic properties of essential oils in the treatment of various pathologies is sought for.

### DESCRIPTION OF THE FIGURES AND EMBODIMENTS

Other advantages and particularities of the invention will appear after reading the detailed description of the embodiments, with reference to the following attached drawings:

FIG. 1 is a schematic diagram of the essential steps of the packaging method according to the invention; and

FIG. 2 schematically illustrates an example of embodiment of the packaging method according to the invention for the production of capsules.

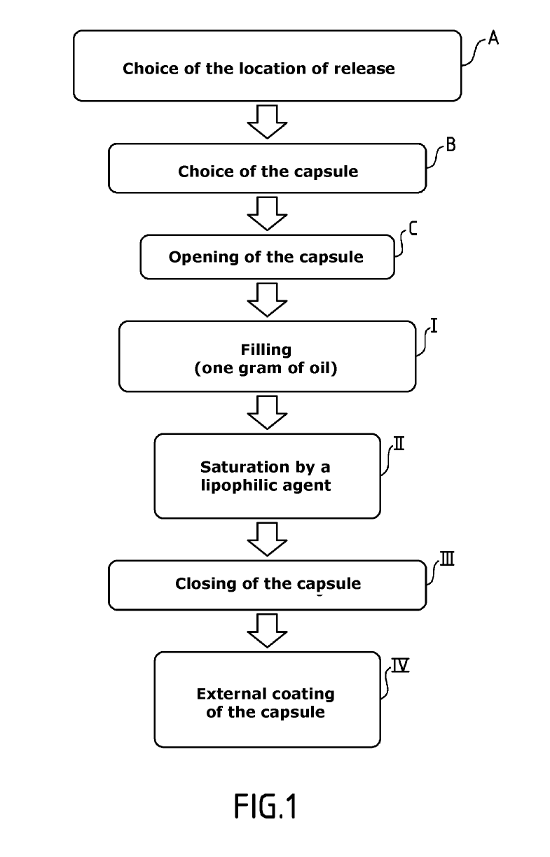

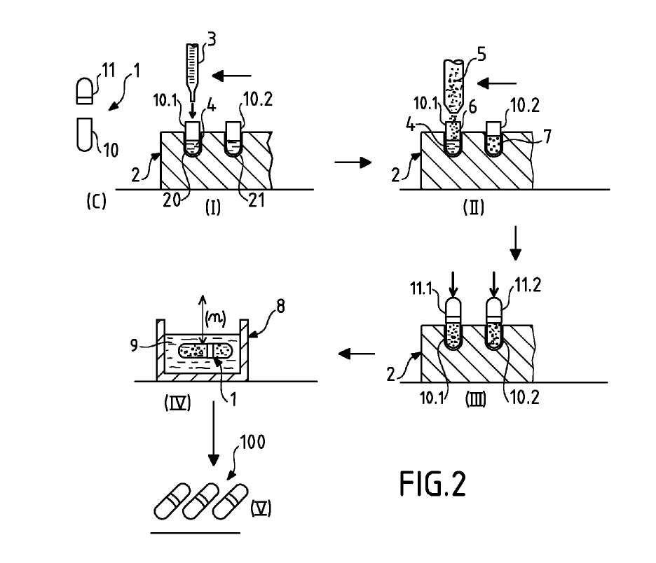

Now, with reference to FIGS. 1 and 2, the implementation of the packaging method according to the invention, based on specifications established for the design of capsules intended to contain a pure essential oil or a complex of essential oils will be described.

A first design step A consists in choosing the place of release of the lipid compound in the body of a patient or animal. This choice will determine the nature of the final coating of the capsules. A capsule is then selected (step B) as the future container of the lipid compound.

Once a capsule 1, previously manufactured according to current industrial standards, has been chosen to contain the lipid compound, it is opened in two parts 10, 11 (step C).

The following steps for packaging the capsules will now be described with reference to FIGS. 1 and 2.

The first parts 10.1, 10.2, . . . , 10.i of empty prefabricated capsules, preferably the longest parts of these capsules, are arranged in cylindrical housings 20, 21, . . . , 2i of suitable shape and arranged in a support 2.

A first packaging step (I) consists in partially filling the first parts of capsule 10.1, 10.2, . . . , 10i with a determined quantity of essential oil or lipid compound 4 poured from a filling device 3 movably controlled above support 2 in an automated version of the packaging system according to the invention. As a non-limiting example, a mass of one gram of oil can be poured into the first parts 10.1, 10.2, . . . , 10i of the open capsules.

In a second packaging step (II), a lipophilic agent 6 is poured from a motion-controlled pouring device 5 into the first part 10.1 of a capsule until the oil or lipid compound 4 already present is saturated.

Slow filling ensures that the oil is absorbed without overflowing the container. As a non-limiting example, the capsule finally reaches a mass of about 1.5 grams.

By mixing lipid compound 4 and lipophilic agent 5, a pasty substance is obtained with a variable dissolution rate depending on the type of lipophilic agent chosen.

This is followed by a step (III) during which the capsules thus filled are closed by means of their respective second closing parts 11.1, 11.2, . . . , 11.i which are fitted to in their respective first parts 10.1, 10.2, . . . , 10.i.

Given the high concentration of the oils used, which ensures their therapeutic effect, the capsule must then undergo a coating treatment in order to strengthen the protection of the envelope and ensure optimal and durable imperviousness, which ensures commercial feasibility.

After filling the capsule with the essential oil/absorbent mixture and closing it, a peripheral coating of the capsule will ensure its imperviousness over time, hermeticity and mechanical strength. This coating also allows the dissolution rate to be chosen, and therefore also the place of dissolution within the digestive tract.

The capsules thus filled and closed are subjected, during a coating (IV) step, either to several (n) successive dips in a bath filled with a polymer component in the liquid phase, or to several contacts with these polymers, for example, and as a non-limiting example, by spraying, in order to externally coat the capsule, which will provide an adjustable function of releasing the active principle contained in the capsule according to the pH of the surrounding environment. It is thus possible to predetermine, within the human or animal body ingesting this capsule, the place where the lipid compounds will be released.

At the end of this coating step (IV), capsules that are now operational have been obtained, which will then be packaged in packages 100 adapted to their distribution (step V).

The selection of the different components used in the packaging method according to the invention will now be described in more detail.

To make the capsule shell for the concentrated and stabilized oil preparations, in practice, empty capsules already manufactured and marketed by specialized laboratories can be chosen. The capsules are enteric and preferably of white color and opaque to avoid deterioration over time by prolonged exposure to light. The size of these capsules must allow them to be filled with a volume corresponding to one gram of oil plus the retained lipophilic stabilizing agent. The size chosen is therefore of the known type “000”, which corresponds to an inner volume of 1.37 ml.

Elements involved in the choice of the food-grade lipophilic absorbent agent will now be considered. This choice will depend on the objective for a more or less rapid release and absorption rate, in a context of packaging and coating one gram of oil or lipid compound to be slowly dissolved in the digestive tract

As a non-limiting example, two food absorbents were used: food-grade clay and tapioca.

Food-grade clay is gastroresistant. It ensures the production of a thick mixture with a slow dissolution rate. The mixture of clay and oils so obtained is thus released very slowly and will therefore allow release in the posterior or terminal part of the intestinal tract.

Tapioca is a starch used in cooking, produced from the roots of cassava, dried and then pulverized. It ensures the production of a softer mixture with a faster dissolution rate. The tapioca and oil mixture so obtained is thus released more quickly and will therefore allow release in the anterior part of the intestinal tract.

The coating of the capsule previously filled with the absorbent+oil mixture and closed again can be done, for example, with a specific polymer such as EUDRAGIT® marketed by EVONIK INDUSTRIES. Different products of this type allow the dissolution site of the capsule according to a pH level that triggers the dissolution of the coating to be adjusted.

The chemical composition of these products is that of copolymers of methacrylic acid and ester of this acid. Three derivatives are particularly well known: EUDRAGIT L, S and L30D. They differ by their content in methacrylic groups.

Hence, EUDRAGIT L® is rich in these groups: its dissolution pH is 6. It is therefore more suitable for coating formulations that must release the active ingredient at the level of the duodenum.

On the contrary, EUDRAGIT S® contains few of such groups. This product only dissolves at pH 7 and above. It is therefore chosen for forms that are expected to act at posterior levels of the digestive tract.

The more baths in this coating polymer, the thicker the coating layer is and the more resistant the capsule is to acid attack.

Since these embodiments are in no way limiting, it will be possible to consider variants of the invention comprising only a selection of characteristics described or illustrated, subsequently isolated from the other characteristics described or illustrated (even if this selection is isolated within a sentence comprising these other characteristics), if this selection of characteristics is sufficient to confer a technical advantage or distinguish the invention from the prior art. This selection shall include at least one characteristic which is preferably functional without structural details, and/or with only part of the structural details if this part alone is sufficient to confer a technical advantage or differentiate the invention from the prior art.

Of course, the invention is not limited to the examples just described and many adjustments can be made to these examples without going beyond the scope of the invention.

Of course, the different characteristics, forms, variants and embodiments of the invention may be associated with each other in various combinations to the extent that they are not incompatible with or exclusive of one another. In particular, all the variants and embodiments described above can be combined with each other.

1. A method for packaging a lipid compound for ingestion by a human or animal body, comprising the following steps:
filling (I) a first part of a prefabricated capsule previously opened in two parts, with a predetermined volume of a lipid compound;
inserting (II) a food-grade lipophilic absorbent into the lipid compound within said first part, so as to produce, after absorption of said lipid compound, a core in the form of a pasty mixture providing a predetermined dissolution rate,
closing (III) said capsule by fitting the second part on the first part containing said core; and
coating (IV) said capsule thus closed, with a polymer, so as to allow subsequent release of the lipid compound at a predetermined releasing site within said human or animal body.

2. The method according to claim 1, wherein the coating step (IV) comprises one or more successive treatments of the capsule, the said treatment(s) comprising the application of a coating polymer on the periphery of said capsule, the number (n) of said treatments determining the thickness of the coating layer and thus the resistance of the capsule to acid attacks.

3. The method according to claim 1, wherein the coating polymer has a dissolution pH of substantially 6, whereby the capsule is provided for releasing the lipid compound substantially in the duodenum of a human or animal body.

4. The method according to claim 1, wherein the coating polymer has a dissolution pH substantially greater than or equal to 7, whereby the capsule is provided for releasing the lipid compound in posterior regions of the digestive tract of a human or animal body.

5. The method according to claim 1, wherein the lipophilic absorbent comprises food clay, so that the mixture thus obtained is thick and provides for a very slow release of the lipid compound.

6. The method according to claim 1, wherein the lipophilic absorbent comprises tapioca, so that the mixture thus obtained is flexible and provides for a rapid release of the lipid compound.

7. The method according to claim 1, wherein the lipid compound comprises a pure essential oil.

8. The method according to claim 1, wherein the lipid compound comprises a mixture of essential oils.

9. A system for packaging a lipid compound intended for ingestion by a human or animal body, carrying out the method according to any one of the foregoing claims, comprising:
means for filling a first part of a prefabricated capsule previously opened in two parts, with a predetermined volume of a lipid compound;
means for introducing a food-grade lipophilic absorbent into the lipid compound within said first part, so as to provide, upon absorption of said lipid compound, a core in the form of a pasty mixture providing for a predetermined dissolution rate,
means for closing said capsule by fitting the second part on the first part containing said core; and
means for coating said closed capsule with a polymer provided for releasing the lipid compound at a predetermined release location.

10. The system according to claim 9, wherein the coating means are arranged to carry out one or more successive treatments of the capsule, the said treatment(s) comprising the application of a coating polymer on the periphery of said capsule, the number of said treatments determining the thickness of the coating layer and thus the resistance of the capsule to acid attacks.

11. (canceled)

12. (canceled)

13. A functional food or a medicament, comprising a closed capsule containing a pasty mixture, the pasty mixture consisting in a pure essential oil or a mixture of essential oils absorbed in an absorbent.

14. The functional food or medicament according to claim 13, wherein the absorbent comprises or consists in tapioca.

15. The functional food or medicament according to claim 14, wherein the closed capsule is configured to release the pure essential oil or the mixture of essential oils in the intestine.

16. The functional food or medicament according to claim 13, wherein the absorbent comprises or consists in food clay.

17. The functional food or medicament according to claim 14, wherein the closed capsule is configured to release the pure essential oil or the mixture of essential oils in the intestine.
